(19) United States
(12) Patent Application Publication
(10) Pub. No.: US 2007/0141175 A1
(43) Pub. Date: Jun. 21, 2007

(54) Chemotherapy adjuvant

(76) Inventors: Mark Gostine, Grand Rapids, MI (US); Larry Pawl, Grand Rapids, MI (US)

Correspondence Address: JONATHAN P. O'BRIEN, MILLER CANFIELD PADDOCK and;STONE, 444 West Michigan Avenue, Kalamazoo, MI, 49007, US

(21) Appl. No.: 11/642,218

(22) Filed: Dec. 20, 2006

Related U.S. Application Data
(60) Provisional application No. 60/751,789, filed on Dec. 20, 2005.
(60) Provisional application No. 60/756,278, filed on Jan. 5, 2006.

(51) Int. Cl. A61K 33/04; A61K 31/385; A61K 31/375; A61K 31/198; A61K 31/205

(52) U.S. Cl. 424/702; 514/474; 514/554; 514/440; 514/562

(57) ABSTRACT

The present invention provides chemotherapy adjuvants for reducing the severity of side effects caused by one or more chemotherapy agent(s), methods of reducing the severity of chemotherapy agent side effects, and methods of treating cancer.

### CLAIM OF PRIORITY

This patent application claims the benefit of U.S. Provisional Patent Application Ser. No. 60/751,789, filed on Dec. 20, 2005 and U.S. Provisional Patent Application Ser. No. 60/756,278, filed on Jan. 5, 2006, both of which are hereby incorporated by reference.

### FIELD OF THE INVENTION

The invention relates to chemotherapy adjuvants that reduce the severity of side effects caused by chemotherapy agents, and methods of reducing the side effects of chemotherapy agents.

### BACKGROUND OF THE INVENTION

Each year, thousands of people are diagnosed with some form of cancer. A diagnosis of cancer traditionally involves serious health implications. Cancer can cause disfigurement, chronic or acute pain, lesions, organ failure, or even death. Commonly diagnosed cancers include breast cancer, lung cancer, melanoma, non-Hodgkin's lymphoma, leukemia, endometrial cancer, colon and rectal cancer, prostate cancer, and bladder cancer. Traditionally, many cancers (e.g., breast cancer, leukemia, lung cancer, or the like) are treated with surgery, chemotherapy, radiation, or combinations thereof. Chemotherapy agents used in the treatment of cancer are known to produce several serious and unpleasant side effects in patients. For example, some chemotherapy agents cause neuropathy, nephrotoxicity (e.g., hyperlipidemia, proteinuria, hypoproteinemia, combinations thereof, or the like), stomatitis, mucositisemesis, alopecia, anorexia, esophagitis amenorrhoea, decreased immunity, anaemia, high tone hearing loss, cardiotoxicity, fatigue, neuropathy, or combinations thereof.

### SUMMARY OF THE INVENTION

The inventors have recognized solutions to one or more problems above by providing chemotherapy adjuvants that promote good health and reduce the severity of unpleasant side effects caused by chemotherapy agents. Moreover, this invention provides methods of reducing the severity of side effects caused by chemotherapy agents. Chemotherapy adjuvants of the present invention are formulated with vitamins, minerals, amino acids, coenzymes or cofactors, and other ingredients that promote health. Methods of reducing the severity of the side effects associated with chemotherapy agents include administering to a patient, 1-5 times daily, a single dosage unit form of a chemotherapy adjuvant prior to starting a course of chemotherapy, during the course of chemotherapy, and continuing after the course of chemotherapy has ended.

One aspect of the present invention provides chemotherapy adjuvant compositions useful for reducing the toxic side effects of a course of chemotherapy comprising from about 20 mg to about 150 mg of vitamin C, from about 20 μg to about 120 μg of selenium, and more than about 200 mg of L-carnitine.

Another aspect of the present invention provides methods of reducing the side effects of a course of chemotherapy involving administering to a patient a first chemotherapy adjuvant composition 1-5 times a day prior to and during a course of chemotherapy; and administering to the patient a second chemotherapy adjuvant composition 1-5 times a day after the course of chemotherapy has concluded; wherein the first chemotherapy adjuvant comprises from about 20 mg to about 150 mg of vitamin C, from about 20 μg to about 120 μg of selenium, and more than about 200 mg of L-carnitine; and the second chemotherapy adjuvant comprises from about 20 mg to about 150 mg of vitamin C, from about 20 μg to about 120 μg selenium, more than about 200 mg of L-carnitine, and from about 100 mg to about 600 mg of cysteine.

The following modifications apply to all of the abovementioned aspects of the present invention. Furthermore, chemotherapy adjuvant compositions can comprising from about 40 mg to about 120 mg of vitamin C. Chemotherapy adjuvant compositions can comprise from about 40 mg to about 80 mg of vitamin C. Chemotherapy adjuvant compositions can comprising from about 30 μg to about 110 μg of selenium, or chemotherapy adjuvant compositions can comprise from about 30 μg to about 60 μg of selenium. Chemotherapy adjuvant compositions can further comprise more than about 250 mg of L-carnitine. Chemotherapy adjuvant compositions can also comprise α-lipoic acid, cysteine, or a combination thereof. In fact, chemotherapy adjuvant compositions can comprise from about 100 mg to about 600 mg of cysteine or even from about 150 mg to about 250 mg of cysteine. Chemotherapy adjuvant compositions can comprise from about 100 mg to about 300 mg of α-lipoic acid. Some chemotherapy adjuvant compositions comprise a binding agent, a colorant, a fragrance, a sweetener, or a flavoring.

In several aspects of the present invention, a course of chemotherapy includes administering to a patient a chemotherapy agent comprising taxanes, anthracyclines, platinums, vincalkaloids, or combinations thereof.

Another aspect of the present invention provides chemotherapy adjuvant compositions comprising from about 9.3 wt % to about 11.2 wt % of vitamin C, from about 0.50 wt % to about 1 wt % of selenium, and from about 45 wt % to about 70 wt % of L-carnitine. These chemotherapy adjuvant compositions can further comprise from about 0 wt % to about 20 wt % of cysteine, from about 2 wt % to about 30 wt % of rice flour, from about 1.0 wt % to about 1.5 wt % of vegetable stearate.

Another aspect of the present invention provides chemotherapy adjuvant compositions for reducing the toxic side effects of a course of chemotherapy comprising a single dosage unit form including about 10.42 wt % of vitamin C, about 0.81 wt % of selenium, about 60.36 wt % of L-carnitine, about 1.18 wt % of vegetable stearate, and about 27.23 wt % of rice flour.

Another aspect of the present invention provides chemotherapy adjuvant compositions for reducing the toxic side effects of a course of chemotherapy comprising a single dosage unit form including about 10.13 wt % of vitamin C, about 0.79 wt % of selenium, about 53.3 wt % of L-carnitine, about 14.29 wt % of α-lipoic acid, about 17.86 wt % of cysteine, about 1.14 wt % of vegetable stearate, and about 2.49 wt % of rice flour.

In any of the abovementioned aspects, chemotherapy adjuvant compositions can be formulated as a tablet, a capsule, a powder, an emulsion, or a solution. Furthermore, chemotherapy adjuvant compositions can be administered to a patient 1-3 times a day for up to 2 weeks prior to starting the course of chemotherapy. Other chemotherapy adjuvant compositions can be administered to a patient 1-3 times a day starting on the day after the patient has concluded the course of chemotherapy. A patient can be administered a first chemotherapy adjuvant composition, a second chemotherapy adjuvant composition, or both, 2 times a day. The first chemotherapy adjuvant composition can comprise from about 40 mg to about 120 mg of vitamin C, and the second chemotherapy adjuvant composition can comprise from about 40 mg to about 150 mg of vitamin C, or the first chemotherapy adjuvant composition can also comprise from about 40 mg to about 80 mg of vitamin C, and the second chemotherapy adjuvant composition can comprise from about 40 mg to about 120 mg of vitamin C. The first chemotherapy adjuvant composition can comprise from about 30 μg to about 60 μg of selenium, and the second chemotherapy adjuvant composition comprises from about 30 mg to about 110 μg of selenium. The first chemotherapy adjuvant composition can comprise more than about 250 mg of L-carnitine. The second chemotherapy adjuvant composition comprises from about 150 mg to about 250 mg of cysteine and/or from about 100 mg to about 300 mg of β-lipoic acid.

In another aspect, a patient is administered a first chemotherapy adjuvant composition 2-3 times a day prior to or during a course of chemotherapy wherein the course of chemotherapy includes administering to a patient a chemotherapy agent comprising taxanes, anthracyclines, platinums, vincalkaloids, or combinations thereof, and a second chemotherapy adjuvant starting the day after completing a course of chemotherapy.

### DETAILED DESCRIPTION OF THE INVENTION

The present invention provides chemotherapy adjuvants for reducing the severity of side effects caused by one or more chemotherapy agent(s) and methods of reducing the severity of chemotherapy agent side effects.

A. Definitions

As used herein, a “chemotherapy agent” is a cytotoxic drug or cytotoxic mixture of drugs that that are intended to destroy malignant cells and tissues. Examples include taxanes, anthracyclines, platinums, vincalkaloids, or combinations thereof.

As used herein, a “course of chemotherapy” is a schedule or regimen for administering chemotherapy agents to a cancer patient.

As used herein, a “chemotherapy adjuvant” or “chemotherapy adjuvant composition” is an additional treatment used to increase the effectiveness of the primary chemotherapy agent(s) (e.g., by reducing the severity of side effects, increasing the effect(s) of the chemotherapy agent(s), or a combination thereof).

The expression “single dosage unit form” as used herein refers to a physically discrete unit of chemotherapy adjuvant appropriate for the patient to be treated.

The term “patient” as used herein, means an animal, preferably a mammal, and most preferably a human.

As used herein a “therapeutically effective amount” or “pharmaceutically acceptable amount” is an amount of a compound that when administered to a patient achieves some desired effect.

As used herein, a “binder” or “binding agent” is a non-toxic compound that is used to improve the strength of a solid pharmaceutical formulation. Examples of binders include hydroxypropyl cellulose, rice flour, vegetable stearate, acacia, carboxymethylcellulose sodium, dextrin, ethylcellulose, gelatin, glucose, guar gum, hydroxyethyl cellulose, hydroxypropyl methylcellulose, methylcellulose, polymethacrylates, povidone, pregelatinized starch, sodium alginate, zein, or any combination thereof.

B. Chemotherapy Adjuvants

A chemotherapy adjuvant of the present invention includes vitamin C, selenium, and L-carnitine. The vitamin C (e.g., ascorbic acid), selenium, and L-carnitine can be present in any therapeutically acceptable amount (e.g., an amount that does not generate a toxic response in a patient). In several embodiments, the vitamin C, selenium, and L-carnitine are present in a therapeutically effective amount. Moreover, the ingredients (e.g., vitamin C, selenium, and L-carnitine) can be present in any suitable form (e.g., the ingredients are bioavailable, and non-toxic when by themselves or combined with other ingredients). Moreover, vitamin C, selenium, and L-carnitine can be administered simultaneously (e.g., as a mixture in a single capsule or tablet), concurrently (e.g., as a plurality of capsules or tablets administered at about the same time), or separately (as a plurality of capsules or tablets administered at different times).

In one embodiment, the chemotherapy adjuvant is apportioned into single dosage unit forms (e.g., capsules, tablets or pills, powders, liquid dosage forms, combinations thereof, or the like). A single dosage unit form can comprise any amount of vitamin C, selenium, and L-carnitine that does not cause a toxic response in the patient.

In several embodiments, a single dosage unit form of a chemotherapy adjuvant of the present invention comprises from about 100 mg to about 150 mg of vitamin C. In another example, a single dosage unit form of a chemotherapy adjuvant comprises from about 110 mg to about 140 mg of vitamin C. In another example, a single dosage unit form comprises from about 120 mg to about 130 mg of vitamin C. In still another example, a single dosage unit form of an adjuvant comprises from about 122 mg to about 128 mg (e.g., about 125 mg) of vitamin C.

In several other embodiments, a single dosage unit form of a chemotherapy adjuvant of the present invention comprises from about 20 mg to about 150 mg of vitamin C. For example, a single dosage unit form comprises about 20 mg to about 120 mg (e.g., from about 30 mg to about 70 mg, from about 40 mg to about 60 mg, from about 60 mg to about 80 mg, from about 80 mg to about 120 mg, from about 90 mg to about 110 mg, from about 95 mg to about 105 mg, about 50 mg, about 75 mg, or about 100 mg) of vitamin C. In alternative examples, a chemotherapy adjuvant comprises from about 40 mg to about 150 mg (e.g., from about 40 mg to about 80 mg, or from about 40 mg to about 120 mg) of vitamin C.

Vitamin C can be present in a chemotherapy adjuvant of the present invention in any suitable form. In several examples, vitamin C is present in a water soluble form; however, in several other examples, vitamin C is present in a lipid soluble form. In several examples, vitamin C is present in the form of sodium ascorbate, calcium ascorbate, potassium ascorbate, magnesium ascorbate, zinc ascorbate, molybdenum ascorbate, chromium ascorbate, manganese ascorbate, dehydroascorbate (oxidized ascorbic acid), calcium threonate, xylonite, lyxonate, ascorbyl palmitate, combinations thereof, or the like. In some embodiments, vitamin C is present in the form of ascorbic acid.

In other embodiments, a single dosage unit form of a chemotherapy adjuvant of the present invention comprises from about 10 μg to about 100 μg of selenium. In other examples, a single dosage unit form of the present invention comprises from about 20 μg to about 90 μg. In several embodiments, a single dosage unit form includes from about 40 μg to about 60 μg (e.g., from about 48 μg to about 52 μg, or about 50 μg).

In other examples, a single dosage unit form of a chemotherapy adjuvant of the present invention comprised from about 20 μg to about 120 μg of selenium. In other embodiments, a single dosage unit form comprises from about 30 μg to about 110 μg (e.g., from 20 μg to about 60 μg, from about 30 μg to about 60 μg, from about 35 μg to about 45 μg, or about 40 μg) of selenium or from about 80 μg to about 120 μg (e.g., from about 90 μg to about 110 μg, from about 95 μg to about 105 μg, or about 100 μg) of selenium.

A chemotherapy adjuvant of the present invention comprises selenium in any suitable form. In several examples, a chemotherapy adjuvant of the present invention includes selenium in the form of L-selenomethionine, selenomethionine, selenium methconine, sodium selenate, sodium selenite, or combinations thereof. In several embodiments, selenium is present in the form of selenium methconine or selenomethionine.

In several embodiments, a single dosage unit form of a chemotherapy adjuvant of the present invention comprises from about 400 mg to about 600 mg of L-carnitine. In other examples, a single dosage unit form of an adjuvant of the present invention comprises from about 450 mg to about 550 mg of L-carnitine (e.g., from about 475 mg to about 525 mg; from about 490 mg to about 505 mg; from about 499 mg to about 501 mg, or about 500 mg).

In alternative embodiments, a single dosage unit form of a chemotherapy adjuvant of the present invention comprises more that about 200 mg of L-carnitine. In other examples, the single dosage unit form comprises more than about 250 mg (e.g., from about 250 mg to about 550 mg, from about 250 mg to about 350 mg, from about 280 mg to about 320 mg, from about 290 mg to about 310 mg, about 300 mg, about 400 mg, or about 500 mg) of L-carnitine.

L-carnitine can be present in chemotherapy adjuvants of the present invention in any suitable form. For example L-acetylcarnitine, acetyl-L-carnitine, L-proprionyl carnitine, L-carnitine fumarate, L-carnitine tartrate, L-carnitine magnesium citrate, combinations thereof, or the like. In several embodiments, L-carnitine is present in the form of L-carnitine tartrate.

Chemotherapy adjuvants of the present invention can optionally comprise α-lipoic acid, cysteine, or both. Single dosage unit forms of chemotherapy adjuvants comprising α-lipoic acid or cysteine can include them in any suitable form. Adjuvants of the present invention comprise an amount of α-lipoic acid and/or cysteine that is therapeutically effective. The adjuvants can also comprise any suitable form of α-lipoic acid and/or cysteine.

In several embodiments, a single dosage unit form of a chemotherapy adjuvant optionally comprises from about 100 mg to about 300 mg of α-lipoic acid. In other embodiments, a single dosage unit form comprises from about 150 mg to about 250 mg of α-lipoic acid. In still other embodiments, a single dosage unit form comprises from about 180 mg to about 220 mg (e.g., from about 190 mg to about 210 mg, from about 199 mg to about 201 mg, or about 200 mg) of α-lipoic acid. In another group of examples, a single dosage unit form of a chemotherapy adjuvant comprised from about 100 mg to about 600 mg (e.g., from 150 mg to about 450 mg, from about 450 mg to about 500 mg, about 200 mg, about 400 mg) of α-lipoic acid. Several alternative embodiments comprise cysteine but do not include α-lipoic acid (e.g., the embodiment is substantially free of α-lipoic acid (e.g., having less than 0.01 wt % of α-lipoic acid)).

α-lipoic acid can be present in chemotherapy adjuvants of the present invention in any suitable form. For example, α-lipoic acid includes R or S enantiomers, or racemic mixtures thereof, or can be present as α-dihydrolipoic acid. In several embodiments, α-lipoic acid can be present as an R enantiomer, a S enantiomer, or a racemic mixture of the two.

In several embodiments, a single dosage unit form of a chemotherapy adjuvant comprises from about 200 mg to about 300 mg of cysteine. For example, a single dosage unit form of a chemotherapy adjuvant comprises about 225 mg to about 275 mg of cysteine, about 240 mg to about 255 mg of cysteine, about 249 mg to about 251 mg of cysteine, or about 250 mg of cysteine. In another group of examples, a single dosage unit form of a chemotherapy adjuvant comprises from about 100 mg to about 600 mg (e.g., from about 150 mg to about 250 mg, from about 350 mg to about 450 mg, about 200 mg, about 400 mg, or about 500 mg) of cysteine.

Cysteine can be present in chemotherapy adjuvants of the present invention in any suitable form. For example cysteine can be present as N-acetylcysteine, L-cysteine HCL anhydrous, L-cystine, or combinations thereof. In other embodiments, cysteine is present as N-acetylcysteine.

In other embodiments, a chemotherapy adjuvant comprises additional vitamins, oils, minerals, combinations thereof, or the like. In several embodiments, a chemotherapy adjuvant comprises additives such as stabilizers, colorants, fragrances, sweeteners, flavors, or the like. For example, several embodiments include one or more stabilizers such as rice flour, magnesium stearate, vegetable stearate, combinations thereof, or the like. Optionally, chemotherapy adjuvants can also comprise gelatin coatings, gelatin capsules, veggie capsules, or combinations thereof.

C. Methods of Use

In yet another aspect, a method for reducing the severity of the side effects of a chemotherapy agent (e.g., neuropathy, nephrotoxicity (e.g., hyperlipidemia, proteinuria, hypoproteinemia, combinations thereof, or the like), stomatitis, mucositisemesis, alopecia, anorexia, esophagitis amenorrhoea, decreased immunity, anaemia, high tone hearing loss, cardiotoxicity, fatigue, combinations thereof, or the like) is disclosed. In certain embodiments, a method of reducing the severity of chemotherapy agent side effects comprises administering to a patient a first single dosage unit form of a first chemotherapy adjuvant 1-5 times a day prior to and during a course of chemotherapy and administering a second single dosage unit form of a second chemotherapy adjuvant 1-5 times a day once the course of chemotherapy has concluded. In several methods, the chemotherapy adjuvant comprises vitamin C, selenium, and L-carnitine. In other methods, the course of chemotherapy includes treating the patient with a chemotherapy agent comprising a taxane, an anthracycline, a platinum, a vincalkaloid, or a combination thereof.

In certain embodiments of the present invention, a single dosage of a chemotherapy adjuvant is administered to a patient 1-5 times a day (e.g., 1, 2, 3, or 4 times a day) before the patient undergoes a course of chemotherapy (e.g., prior to the patient being administered a chemotherapy agent), during the course of chemotherapy, and/or after the course of chemotherapy (e.g., after the patient has been administered the final dosage of a chemotherapy agent in a course of chemotherapy). For example, a patient can be administered an adjuvant 1-5 times a day for more than about 1 hour (e.g., more than about 24 hours, more than about 48 hours, more than about 1 week, more than about 2 weeks, more than about 1 month, more than about 2 months, or more) prior to starting a course of chemotherapy, throughout the course of chemotherapy, and/or for more than 1 day (e.g., 3 days, 1 week, 1 month, 2 months, or more) after the course of chemotherapy has concluded.

In another embodiment, a patient is administered a chemotherapy adjuvant comprising vitamin C, selenium, and L-carnitine 1-3 times a day prior to, during, or after the course of treatment with a chemotherapy agent. In some embodiments, the patient is administered the chemotherapy adjuvant comprising vitamin C, selenium, and L-carnitine prior to and during the course of chemotherapy.

In another embodiment, a patient is administered a chemotherapy adjuvant comprising vitamin C, selenium, L-carnitine, α-lipoic acid, and cysteine 1-3 times a day prior to, during, or after the course of treatment with a chemotherapy agent. In some other examples, a patient is administered a chemotherapy adjuvant comprising vitamin C, selenium, L-carnitine, and cysteine 1-3 times a day prior to, during, or after the course of treatment with a chemotherapy agent.

In several embodiments, the patient is administered a first chemotherapy adjuvant comprising vitamin C, selenium, and L-carnitine prior to and during the course of chemotherapy, and administered a second adjuvant comprising vitamin C, selenium, L-carnitine, α-lipoic acid, and cysteine after the course of chemotherapy has concluded.

Alternatively, the patient is administered a first chemotherapy adjuvant comprising vitamin C, selenium, and L-carnitine prior to and during the course of chemotherapy, and administered a second adjuvant comprising vitamin C, selenium, L-carnitine, and cysteine after the course of chemotherapy has concluded.

In another embodiment, the administration of a chemotherapy adjuvant is terminated when side effects related to the course of treatment with a chemotherapy agent are at least partially alleviated or reduced.

Another aspect of the present invention includes methods of reducing neuropathy comprising administering to a patient, 1-5 times daily, a chemotherapy adjuvant of the present invention under the neuropathy is at least partially reduced.

D. Administrations:

The compounds and compositions, according to the method of the present invention, may be administered using any amount and any route of administration effective for treating or reducing the severity of one or more chemotherapy side effects. In several embodiments, the compounds of the invention are formulated in single dosage unit form for ease of administration and uniformity of dosage.

The chemotherapy adjuvants of this invention can be administered to patients (e.g., humans or other animals) intravenously, orally, rectally, parenterally, intracistemally, intraperitoneally, or via combinations thereof. In certain embodiments, the compounds of the invention may be administered orally or parenterally.

Solid dosage forms for oral administration include, but are not limited to pharmaceutically acceptable tablets, capsules, gel-coated tables, powders, or the like. For example, solid dosage forms can include gelatin capsules or veggie capsules.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, USP and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the vitamin C, selenium, L-carnitine, α-lipoic acid, and/or cysteine can be mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are prepared by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Adjuvants of the present invention can also be combined with foodstuff for oral administration. Adjuvants of the present invention can be combined with a drink powder i.e., one that is reconstituted by adding a liquid, (e.g., an instant shake mix, a flavored juice mix, instant tea, instant coffee, or the like). Adjuvants can also be mixed with drinks such as fruit juice, soy milk, tea, or carbonated beverages (e.g., soda pop or the like). In other embodiments, adjuvants of the present invention are combined with granola bars, fruit bars, or other snack foods.

As described generally above, the compounds of the invention are useful for reducing the severity of neuropathy, nephrotoxicity, stomatitis, mucositisemesis, alopecia, anorexia, esophagitis amenorrhoea, decreased immunity, anaemia, high tone hearing loss, cardiotoxicity, fatigue or combinations thereof caused by a chemotherapy agent.

It will also be appreciated that the chemotherapy adjuvants of the present invention can be employed in combination therapies, that is, the adjuvants can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another agent used to treat the same disorder), or they may achieve different effects (e.g., control of any adverse effects). As used herein, additional therapeutic agents that are normally administered to treat or prevent a particular disease, or condition, are known as “appropriate for the disease, or condition, being treated”. For example, exemplary additional therapeutic agents include, but are not limited to: nonopioid analgesics (indoles such as Etodolac, Indomethacin, Sulindac, Tolmetin; naphthylalkanones such sa Nabumetone; oxicams such as Piroxicam; para-aminophenol derivatives, such as Acetaminophen; propionic acids such as Fenoprofen, Flurbiprofen, Ibuprofen, Ketoprofen, Naproxen, Naproxen sodium, Oxaprozin; salicylates such as Asprin, Choline magnesium trisalicylate, Diflunisal; fenamates such as meclofenamic acid, Mefenamic acid; and pyrazoles such as Phenylbutazone); or opioid (narcotic) agonists (such as Codeine, Fentanyl, Hydromorphone, Levorphanol, Meperidine, Methadone, Morphine, Oxycodone, Oxymorphone, Propoxyphene, Buprenorphine, Butorphanol, Dezocine, Nalbuphine, and Pentazocine). Additionally, nondrug analgesic approaches may be utilized in conjunction with administration of one or more compounds of the invention. For example, anesthesiologic (intraspinal infision, neural blocade), neuro surgical (neurolysis of CNS pathways), neurostimulatory (transcutaneous electrical nerve stimulation, dorsal column stimulation), physiatric (physical therapy, orthotic devices, diathermy), or psychologic (cognitive methods-hypnosis, biofeedback, or behavioral methods) approaches may also be utilized. Additional appropriate therapeutic agents or approaches are described generally in The Merck Manual, Seventeenth Edition, Ed. Mark H. Beers and Robert Berkow, Merck Research Laboratories, 1999, and the Food and Drug Administration website, www.fda.gov, the entire contents of which are hereby incorporated by reference.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention or pharmaceutically acceptable compositions thereof may also be incorporated into compositions for coating an implantable medical device, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition.

### EXAMPLE 1

TABLE 1
Exemplary formulation of chemotherapy adjuvants
of the present invention.

| Ingredients: | Weight Percent Ranges (wt %): |
|---|---|
| Vitamin C (ascorbic acid) | 9.3-11.2 |
| Selenium (selenomethionine) | 0.5-1.0 |
| L-carnitine (tartrate) | 45-70 |
| α-lipoic acid | 0-16 |
| Cysteine (n-acetyl-cysteine) | 0-20 |
| Vegetable Stearate | 1.0-1.5 |
| Rice Flour | 2.0-30.0 |

### EXAMPLE 1a

TABLE 2
Exemplary formulation of one chemotherapy adjuvant
of the present invention.

| Ingredients: | Approximate Weight Percent (wt %): |
|---|---|
| Vitamin C (ascorbic acid) | 10.42 |
| Selenium (selenomethionine) | 0.81 |
| L-carnitine (tartrate) | 60.36 |
| Vegetable Stearate | 1.18 |
| Rice Flour | 27.23 |

### EXAMPLE 1b

TABLE 3
Exemplary formulation of another chemotherapy
adjuvant of the present invention.

| Ingredients: | Approximate Weight Percent (wt %): |
|---|---|
| Vitamin C (ascorbic acid) | 10.13 |
| Selenium (selenomethionine) | 0.79 |
| L-carnitine (tartrate) | 53.30 |
| α-lipoic acid | 14.29 |
| Cysteine (n-acetyl-cysteine) | 17.86 |
| Vegetable Stearate | 1.14 |
| Rice Flour | 2.49 |

### EXAMPLE 2

The method of reducing the side effects of chemotherapy agents can include the following schedule:
a. administering to a patient 2-3 times daily a chemotherapy adjuvant comprising about 125 mg of vitamin C, about 50 μg of selenium, and about 500 mg of L-carnitine starting 2 weeks prior to starting a course of chemotherapy and continuing throughout the course of chemotherapy; and
b. administering to the patient 2-3 times daily a chemotherapy adjuvant comprising about 125 mg of vitamin C, about 50 μg of selenium, about 500 mg of L-carnitine, about 200 mg of α-lipoic acid, and about 250 mg of cysteine starting on the day after the course of chemotherapy concludes and continuing for 1 week or more (e.g., 2 weeks, 1 month, 2 months or more), or until the side effects of the chemotherapy agent(s) are reduced or eliminated altogether.

### EXAMPLE 2a

The method of reducing the side effects of chemotherapy agents can include the following schedule:
a. administering to a patient 2-3 times daily a chemotherapy adjuvant comprising about 50 mg of vitamin C, about 40 μg of selenium, and about 300 mg of L-carnitine starting 2 weeks prior to starting a course of chemotherapy and continuing throughout the course of chemotherapy; and
b. administering to the patient 2-3 times daily a chemotherapy adjuvant comprising about 100 mg of vitamin C, about 100 pg of selenium, about 300 mg of L-carnitine, about 200 mg of α-lipoic acid, and about 250 mg of cysteine starting on the day after the course of chemotherapy concludes and continuing for 1 week or more (e.g., 2 weeks, 1 month, 2 months or more), or until the side effects of the chemotherapy agent(s) are reduced or eliminated altogether.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A chemotherapy adjuvant composition for reducing the toxic side effects of a course of chemotherapy comprising:
from about 20 mg to about 150 mg of vitamin C;
from about 20 μg to about 120 μg of selenium; and
more than about 200 mg of L-carnitine.

2. The chemotherapy adjuvant composition of claim 1, wherein the composition is formulated as a tablet, a capsule, a powder, an emulsion, and a solution.

3. The chemotherapy adjuvant composition of claim 2, comprising from about 40 mg to about 120 mg of vitamin C.

4. The chemotherapy adjuvant composition of claim 3, comprising from about 40 mg to about 80 mg of vitamin C.

5. The chemotherapy adjuvant composition of claim 3, further comprising from about 30 μg to about 110 μg of selenium.

6. The chemotherapy adjuvant composition of claim 5, comprising from about 30 μg to about 60 μg of selenium.

7. The chemotherapy adjuvant composition of claim 5, further comprising more than about 250 mg of L-carnitine.

8. The chemotherapy adjuvant composition of claim 1, further comprising α-lipoic acid, cysteine, or a combination thereof.

9. The chemotherapy adjuvant composition of claim 8, comprising from about 100 mg to about 600 mg of cysteine.

10. The chemotherapy adjuvant composition of claim 9, comprising from about 150 mg to about 250 mg of cysteine.

11. The chemotherapy adjuvant composition of claim 8, comprising from about 100 mg to about 300 mg of α-lipoic acid.

12. The chemotherapy adjuvant composition of claim 1, wherein the composition is formulated as a tablet or a capsule.

13. The chemotherapy adjuvant composition of claim 12, further comprising a binding agent, a colorant, a fragrance, a sweetener, or a flavoring.

14. The chemotherapy adjuvant composition of claim 13, wherein the course of chemotherapy includes administering to a patient a chemotherapy agent comprising taxanes, anthracyclines, platinums, vincalkaloids, or combinations thereof.

15. The chemotherapy adjuvant composition of claim 1, comprising:
from about 9.3 wt % to about 11.2 wt % of vitamin C;
from about 0.50 wt % to about 1 wt % of selenium; and
from about 45 wt % to about 70 wt % of L-carnitine.

16. The chemotherapy adjuvant composition of claim 15, further comprising from about 0 wt % to about 20 wt % of cysteine.

17. The chemotherapy adjuvant composition of claim 16, further comprising at least one binding agent.

18. The chemotherapy adjuvant composition of claim 17, wherein the binding agent comprises rice flour, vegetable stearate, or a combination thereof.

19. The chemotherapy adjuvant composition of claim 18, further comprising
from about 2 wt % to about 30 wt % of rice flour; and
from about 1.0 wt % to about 1.5 wt % of vegetable stearate.

20. A chemotherapy adjuvant composition for reducing the toxic side effects of a chemotherapy agent comprising a single dosage unit form including:
about 10.42 wt % of vitamin C;
about 0.81 wt % of selenium;
about 60.36 wt % of L-carnitine;
about 1.18 wt % of vegetable stearate; and
about 27.23 wt % of rice flour.

21. A chemotherapy adjuvant composition for reducing the toxic side effects of a chemotherapy agent comprising a single dosage unit form including:
about 10.13 wt % of vitamin C;
about 0.79 wt % of selenium;
about 53.3 wt % of L-carnitine;
about 14.29 wt % of α-lipoic acid;
about 17.86 wt % of cysteine;
about 1.14 wt % of vegetable stearate; and
about 2.49 wt % of rice flour.

22. A method of reducing the side effects of course of chemotherapy comprising:
administering to a patient a first chemotherapy adjuvant composition 1-5 times a day prior to and during a course of chemotherapy; and
administering to the patient a second chemotherapy adjuvant composition 1-5 times a day after the course of chemotherapy has concluded;
wherein the first chemotherapy adjuvant composition comprises:
from about 20 mg to about 150 mg of vitamin C, from about 20 μg to about 120 μg of selenium, and
more than about 200 mg of L-carnitine; and
the second chemotherapy adjuvant composition comprises:
from about 20 mg to about 150 mg of vitamin C,
from about 20 μg to about 120 μg selenium,
more than about 200 mg of L-carnitine, and
from about 100 mg to about 600 mg of cysteine.

23. The method of claim 22, wherein the first chemotherapy adjuvant composition or the second chemotherapy adjuvant composition comprises a tablet, a capsule, a powder, an emulsion, or a solution.

24. The method of claim 23, wherein the patient is administered the first chemotherapy adjuvant composition 1-3 times a day for up to 2 weeks prior to starting the course of chemotherapy.

25. The method of claim 24, wherein the patient is administered the second chemotherapy adjuvant composition 1-3 times a day starting on the day after the patient has concluded the course of chemotherapy.

26. The method of claim 25, wherein the patient is administered a first chemotherapy adjuvant composition, a second chemotherapy adjuvant composition or both, 2 times a day.

27. The method of claim 26, wherein the first chemotherapy adjuvant composition comprises about 40 mg to about 120 mg of vitamin C, and the second chemotherapy adjuvant composition comprises from about 40 mg to about 150 mg of vitamin C.

28. The method of claim 27, wherein the first chemotherapy adjuvant composition comprises from about 40 mg to about 80 mg of vitamin C, and the second chemotherapy adjuvant composition comprises from about 40 mg to about 120 mg of vitamin C.

29. The method of claim 28, wherein the first chemotherapy adjuvant composition comprises from about 30 μg to about 60 μg of selenium, and the second chemotherapy adjuvant composition comprises from about 30 mg to about 110 μg of selenium.

30. The method of claim 29, wherein the first chemotherapy adjuvant composition comprises more than about 250 mg of L-carnitine.

31. The method of claim 30, wherein the second chemotherapy adjuvant composition comprises from about 150 mg to about 250 mg of cysteine.

32. The method of claim 31, wherein the second chemotherapy adjuvant composition comprises from about 100 mg to about 300 mg of α-lipoic acid.

33. The method of claim 32, further comprising administering to the patient a first chemotherapy adjuvant composition 2-3 times a day prior to or during a course of chemotherapy wherein the course of chemotherapy includes administering to a patient a chemotherapy agent comprising taxanes, anthracyclines, platinums, vincalkaloids, or combinations thereof.

34. The method of claim 33, further comprising administering to the patient a second chemotherapy adjuvant composition starting the day after completing a course of chemotherapy.
